(19) United States
(12) Patent Application Publication
(10) Pub. No.: US 2026/0207928 A1
(43) Pub. Date: Jul. 23, 2026

(54) MULTI-ELECTRODE LEAD WITH ELONGATED ELECTRODE

(71) Applicant: SORIN CRM SAS, CLAMART (FR)

(72) Inventor: JEAN-FRANÇOIS OLLIVIER, SAINT REMY LES CHEVREUSES (FR)

(21) Appl. No.: 19/142,214

(22) PCT Filed: Dec. 22, 2022
(86) PCT No.: PCT/IB2022/000742

§ 371 Date: Jun. 22, 2025

(51) Int. Cl. A61N 1/05

(52) U.S. Cl. CPC A61N 1/0565; A61N 1/0573

(57) ABSTRACT

The invention relates to a multi-electrode lead device that comprises an interelectrode portion between a distal first electrode and a proximal second electrode, wherein the interelectrode portion comprises an elongated surface electrode for right bundle branch pacing, the elongated surface electrode having a reduced exposed area and at least one non-exposed portion to obtain an increased current density, wherein the elongated surface electrode is connected to a single pacing input terminal of a proximal lead connector. The elongated surface electrode with reduced exposed area enables lower energy consumption by achieving the increased current density and provides an enhanced right bundle branch pacing area for various anatomies.

### FIELD OF THE INVENTION

The invention relates to the field of lead devices (e.g., electrode catheters) for cardiac pacing systems, such as—but not limited to—left bundle branch pacing (LBBP), cardiac resynchronization or tachycardia (“tachy”) systems.

### BACKGROUND OF THE INVENTION

Different electrical activation sequences of cardiac pacing may lead to different mechanical pump efficiencies of a stimulated heart. What is needed is a fast and homogenous contraction of heart ventricles to optimize pump efficiency.

Traditional pacing sites such as the right ventricular apex (RVA) may provide a stable lead position with low displacement rate but are not very effective to optimize left ventricle (LV) contraction (representing about 80% of the heart mass). Long-term RVA pacing may have deleterious effects on left ventricular function by inducing an iatrogenic left bundle branch block (LBBB), which can have strong influences on the left ventricle hemodynamic performances. This observation led to a reassessment of traditional approaches and to a research of alternative pacing sites in order to get to more physiological pattern of ventricular activation and to avoid deleterious effects.

LBBP has emerged as an alternative method for delivering physiological pacing to achieve electrical synchrony of the LV, especially in patients with infranodal atrioventricular block and/or LBBB. Proximal LBBs run through the LV septum and fan out to form a wider target for pacing compared to the His bundle. A technique for LBBP has been developed using a ventricular transseptal approach (i.e., pacing the LV from the RV). LBBP has been reported to offer low pacing thresholds and large R waves, and because the distal conduction system is targeted, has a lower theoretical risk for development of distal conduction block.

After an initial site for an LBBP location at the right surface of the ventricular septum has been determined, the pacing lead (i.e., a helical fixation element or electrode at the lead tip) is screwed into the LV septum, e.g., by puncturing the tissue with the distal tip of the helical fixation element (fixation helix). The LBBP lead depth into the LV septum may be determined by at least one of observing changes in the notch in V1 lead, sheath angiography, fulcrum sign, and impedance monitoring. The pacing lead is slowly progressed into the determined depth (e.g., approximately 6 to 8 mm) by the application of a torque, meanwhile avoiding any perforation of the septum. Finally, LBB capture is confirmed based on acceptable pacing parameters. The confirmation may be based on at least one of a paced morphology of an RBBB pattern, a recording of an LBB potential, a stimulus-peak of the LVAT that shortens abruptly with increasing output or remains shortest and constant at low and high outputs, a selective LBBP and a non-selective LBBP, and a recording of a retrograde His potential or anterograde LBB potential during pacing.

Common features of implantation or placement processes include transvenous access, transseptal placement of the pacing lead into the LV septal subendocardium in the LBB region, and confirmation of capture of the LBB.

However, variations in patient's anatomy may account for certain differences in interventricular thickness of the LV septum, which may vary from about 10 mm to about 20 mm. In addition to uncertain overall septum thickness, the location of the LBB conductive fibers deep inside the septal tissue is also variable between patients, typically located closer to the left ventricle border of the septum, such as about 1-5 mm therefrom. To assure successful left ventricle capture at the lowest capture threshold, the distal electrode of the lead may need to be deployed as close to the LBB fibers as possible, while avoiding the risk of penetrating through the septum and entering the left ventricular cavity.

Moreover, in cases of a combined LBB and RBB pacing, control of both LBB and RBB needs to be achieved by respective correctly positioned LV and RV cathodes. The delivered electrical pulses need to be designed for low energy consumption. From a geometric aspect, this means that at least a section of the RV cathode needs to be as close as possible to the RBB fibers and at least a section of the LV cathode needs to be as close as possible to the LBB fibers.

### SUMMARY OF THE INVENTION

It is an object of the present invention to provide a lead device that is configured to be usable for patients or device recipients despite differences in the anatomy of their septum (especially its thickness) (“various anatomies” hereinafter), while ensuring low energy consumption.

This object is achieved by a lead device as claimed in claim 1.

The proposed lead device comprises an interelectrode portion between the distal first electrode and the proximal second electrode, wherein the interelectrode portion comprises an elongated surface electrode (i.e., with an increased longitudinal length) for right bundle branch pacing, the elongated surface electrode having a reduced exposed area and at least one non-exposed portion to provide an enhanced right bundle branch pacing area at increased current density, and wherein the elongated surface electrode is connected to a only one (i.e., a single) pacing input terminal of a proximal lead connector.

In bipolar leads with first and second electrodes (e.g., an anode and at least one cathode), one electric channel conducts the electrical pacing pulse towards the lead tip and a distal first electrode (e.g., cathode) and the other channel completes the circuit back to the pacemaker via the proximal second electrode (e.g., anode). Myocardial capture requires a minimum local current density (i.e., ratio between pacing current (electrons) and pacing area) at the contact area (exposed area) of the first electrode (cathode). A smaller exposed area leads to an increased current density and also an increased resistance, which preserves battery life as the current flow is reduced.

It is noted that the present invention may as well be used in connection with a unipolar lead. In this case, the second electrode (e.g., anode) may be provided at or may correspond to a housing of a pacing device (e.g., an implantable pacemaker) and the interelectrode portion may range from the first electrode to the pacing device. In this case, a connector (e.g., an IS1 connector) with a single connection electrode or terminal can be used at the proximal end of the lead device.

Accordingly, the elongated surface electrode with a reduced exposedarea is designed for low energy consumption by achieving the increased current density and provides an enhanced right bundle branch pacing area. From a geometric aspect this means that, for various anatomies of the septum, at least a section of the elongated RV cathode can be placed close to the RBB fibers, while at least a section of the LV cathode is placed close to the LBB fibers. Therefore, there is no need for multiple lead models for different anatomies and/or pre-operation examination to measure the septum wall thickness.

The substrate or body of the elongated surface electrode (i.e., a combination of pacing surface and insulated surface) can be configured as a single physical component to maintain a lead design with desired change of stiffness (stiffness gradient) in an area to be inserted into the septum. A change in stiffness leads to a change in flexion of the lead tip and an increased risk of fracture. A plurality of pacing surfaces can thus be arranged (e.g., embedded) on the elongated surface electrode, which may all be electrically connected together and connected to one unique terminal or electrode of a connector at the proximal end of the lead device. As it can be structured as a single mechanical element, it can be configured to provide a continuous stiffness of a desired amount that protects the lead tip. If there were many separate electrodes, this would create a (sudden) stiffness gradient at each separate electrode, which might harm the lead tip during the puncturing process, and this is avoided. Also thereby, the total pacing surface of the elongated surface electrode can be spread over a longer axial distance to maintain good electrical performance by at least one of increasing the probability of having some portion of the elongated surface electrode as close as possible to the tissue area to be stimulated (e.g., the RBB) for various anatomies (e.g., thicknesses of the septum) and maintaining a desired cumulated reduced pacing surface (e.g., 2 to 8 mm²). The pacing surface can be continuous or discontinuous along the axial length of the elongated surface electrode, which may thus consist of one or more separated pacing surface areas that may be electrically connected together.

According to a first option, the non-exposed portion may comprise an electrically insulating cover. The electrically insulating cover serves to reduce the exposed area of the elongated surface electrode to thereby provide an elongated electrode which is better adapted to various anatomies while providing an increased current density for lower energy/battery consumption.

According to a second option, the electrically insulating cover may be arranged in a recess of the elongated surface electrode. Thus, the elongated surface electrode with reduced exposed area can be produced in a reliable and robust manner by having the electrically insulating cover embedded in the recess.

According to a third option, the elongated surface electrode may comprise a helical electrode pattern wound around a lead body of the lead device. This option provides a straight-forward way of reducing the size of the exposed area by simply arranging a helical electrode pattern around the lead body of the lead device in the interelectrode portion.

According to a fourth option, the helical electrode pattern may be placed around or at least partially embedded in an isolating coating of the lead body. The isolating coating ensures that the non-covered portions of the body of the lead device are isolated and thereby non-exposed.

According to a fifth option, the non-exposed portion may comprise at least one cut-out portion that is not covered by the electrically insulating cover. Thus, the elongated surface electrode can be manufactured as a single cylindrical element and the reduced exposed area can be introduced by simply cutting out desired portions (e.g., by laser ablation) to increase the current density now provided by the electrode that consequently has a smaller exposed surface area, and thereby reduce energy/battery consumption.

According to a sixth option, a plurality of the cut-out portions may be distributed over the surface of the elongated surface electrode. This measure ensures, that the current density is more equally distributed over the surface of the elongated surface electrode.

According to a seventh option which can be combined with any one of the first to sixth options, the distal first electrode may be formed by a fixation helix. Thereby, the first electrode can be used to screw the lead tip into the tissue of the septum.

According to an eighth option which can be combined with any one of the first to seventh options, the proximal second electrode may be an anode. Thereby, all electrodes can be provided at the lead tip and can be easily connected to a proximal connector (e.g., a standard IS4 connector).

According to a ninth option which may be combined with the seventh or eighth option, a ratio between a first outer diameter of the fixation helix and a second outer diameter at a distal end of the interelectrode portion may be set between 0.8 and 1, the first outer diameter may be set between 1 and 1.8 mm, the length of the elongated surface electrode may be set between 7 and 11 mm, and the length of the fixation helix may be set between 1.5 and 5 mm. These dimensions facilitate the screwing process of the lead tip into the tissue.

According to a tenth option which can be combined with any one of the first to ninth options, the interelectrode portion may have a conical shape. The conical shape reduces the resistance of the lead tip when entering the tissue during the screwing process.

According to an eleventh option which can be combined with any one of the first to tenth options, the body of the lead device may have a coradial structure. The coradial structure enables a lead design that is less bulky and has less stiffness.

According to a twelfth option which can be combined with any one of the first to eleventh options, the axial distance between the distal end of the fixation helix and the distal end of the elongated surface electrode my range from 7 to 12 mm, the axial length of the elongated surface electrode may range from 7 to 11 mm, and the axial distance between the distal end of the elongated surface electrode and the distal end of the proximal second electrode may range from 10 to 20 mm.

According to a thirteenth option which can be combined with any one of the first to twelfth options, the elongated surface electrode may be configured to obtain a size of a resultant limited active electrode surface within a range of 2 to 8 mm². Thereby, good electrical performance can be achieved, while preserving current consumption and thus device longevity.

It shall be further understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically a heart with a lead device placed for ventricular transseptal LBB pacing;

FIG. 2 shows schematically a side view of a lead device according to a first embodiment with a first cathode formed by a fixation helix and a second cathode formed by an elongated electrode structure with outer coverage for limited exposure;

FIG. 3 shows schematically a cross sectional view of the elongated electrode structure of the first embodiment;

FIG. 4 shows schematically a side view of a lead device according to a second embodiment with a first cathode formed by a fixation helix and a second cathode formed by an elongated helical electrode structure wound around a body of the lead device for limited exposure;

FIG. 5 shows schematically a cross sectional view of the elongated helical electrode structure of the second embodiment; and

FIG. 6 shows schematically a side view of a lead device according to a third embodiment with a first cathode formed by a fixation helix and a second cathode formed by an elongated electrode structure with cut-out portions for limited/reduced exposure.

### DETAILED DESCRIPTION OF EMBODIMENTS

Various embodiments of the present invention are now described based on an improved lead device (e.g., electrode catheter) with fixation helix. Although the present invention is particularly advantageous within the context of transseptal pacing such as LBBP, the invention is not limited thereto and may also be used in connection with other pacing types and/or sites for other applications that require placement of a lead device within a body tissue.

It is noted that throughout the present disclosure only those elements, portions, components and/or devices that are relevant for the proposed lead device and placement operation are shown in the accompanying drawings. Other elements, portions, components and/or devices have been omitted for reasons of brevity. Furthermore, components designated by same reference signs or numbers are intended to have the same or at least a similar function, so that their function is not described again later.

Furthermore, throughout the present disclosure, “proximal” and “distal” are terms that are used to indicate distances from an operating end (reference point) of the lead device, where the physician or other user controls the screwing process. Proximal is closer to the operating end, while distal is further away (at a greater distance) from the operating end.

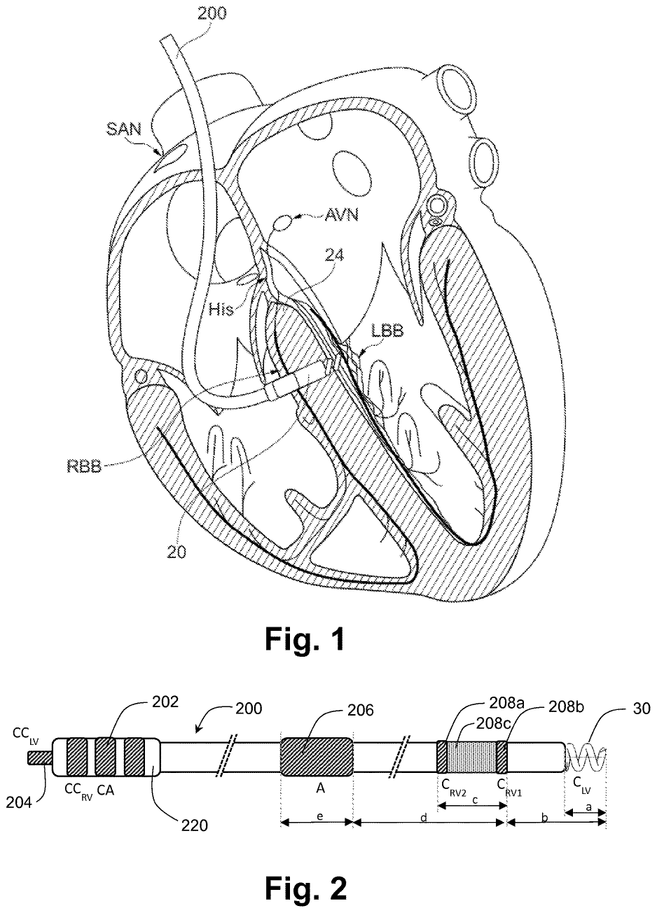

FIG. 1 shows schematically a heart with an inserted lead device 200, where the pacing lead tip 20 is placed for ventricular transseptal LBBP. Thereby, the LV can be paced from the RV by a ventricular transeptal approach as a guide for catheter delivery. The placement of the pacing lead tip 20 may be performed based on the procedure explained above. LBBP may be defined as capture of the LBB (i.e., left bundle trunk or its proximal fascicles/fibers), usually with septal myocardium capture at low output (e.g., <1.0 V/0.4 ms).

In a normal cardiac function, the heartbeat starts in the heart itself due to the sinoatrial node (SAN) which is found in the top of the right atrium (RA) and sets the rate at which the heart contracts. It sends out electrical impulses that are carried through the muscular walls of both atria. These impulses cause atrial systole. The impulse is then passed to another node within the heart-the atrioventricular node (AVN). This node is in the lower part of the RA. Once the impulse from the SAN reaches the AVN the impulse is passed to conducting fibers which travel down the central wall of the heart. The impulse then splits and travels up the LV and RV causing them to contract simultaneously (ventricular systole).

Important elements of the conduction system of the heart are found within the septum 24. The His bundle travels in the subendocardium down the right side of the septum 24 for about 1 cm before dividing into the LBB and RBB. The RBB continues down the right side of the septum 24, while the LBB crosses to the left side and splits into anterior and posterior divisions.

Under normal circumstances, excitation from the SAN controls the heart rhythm. An abnormality in the sinus rhythm leads to arrhythmia, which refers to abnormalities in the rate, rhythm, site of origin, and conduction of the cardiac electrical pulse. When disorders occur in specific intraventricular conduction fibers, the repolarization wave must then travel through the slower muscle-muscle conduction to reach the ventricles. Classic disorders related to conditions that involve different conduction bundle branches include LBBB and RBBB. An electrocardiogram (ECG), as obtained from an inserted lead device, can be used to measure and record cardiac electrical activities and thus can provide important information on cardiac functions. The ECG has been used as a standard diagnostic tool to analyze arrhythmia.

In embodiments, the lead tips of pacing or tachy leads are designed to avoid a risk of perforation of the septum. They may also be equipped with a soft tip (made of e.g. silicone) to increase a stop surface. That is, when the helical fixation element or electrode (called “helix” hereinafter) is engaged with (e.g., screwed into) the (cardiac) issue, this tissue is pushed against the soft tip to stop the helix from rotation and further progression within the tissue. The length of the helix may be limited, e.g., to an active length of about 2 mm. The lead and/or the helix need to be designed for optimizing required energy/force for puncturing and for allowing a well-controlled and safe progression without increasing complexity of the lead.

Furthermore, according to embodiments, the body of the lead device may be configured to improve slipperiness of a contact with a guiding catheter used for guiding the lead device (e.g., through a blood vessel) to a target area. This can be achieved by using e.g. a polyurethane (PU) material with reduced diameter to allow an advancement progress of the lead body through the guiding catheter and the lead tip 20 through the septum 24 with limited effort.

Suitable designs of lead devices according to embodiments may have a multi-lumen, coaxial and coradial structure, both as tachycardia leads or bradycardia leads, and a central lumen for a stylet passage may be provided. Coaxial leads have an inner conductor that extends down the length of the lead to the tip electrode (helix), the cathode, arranged in a coil configuration that provides a central lumen e.g. to allow for passage of a stylet at implantation.

Coradial bipolar leads address some of the disadvantages of coaxial leads with respect to the bulk and stiffness of their four-layer design, by providing a new conductor and insulator technology where a single coil extends down the length of the lead (again with a central lumen to allow for stylet insertion) and consists of two parallel, alternating conductor strands, one of which connects to the cathode and the other to the anode. Each conductor strand may be individually coated with a bonded layer of e.g. ethylene tetrafluoroethylene (ETFE) fluoropolymer insulation that serves to insulate each strand from the other, despite being intertwined. The single, two-component coil may be surrounded by a single, outer insulation covering.

The multi-lumen or coaxial or coradial leads may optionally comprise a fixed, non-retractable helix to minimize size. However, a retractable helix may also be used in connection with the described embodiments.

Furthermore, the proposed multi-electrode lead device according to embodiments may be configured to provide improved torquability, i.e., an ability to transmit torque safely and accurately to the helix (e.g., full lead body torque) and stylet-driven compatibility to ease the handling (e.g., by push transmission). In an example, a coradial lead with compatible screwing stylet (screwdriver stylet) may be provided.

The following embodiments of the proposed multi-electrode lead device are configured to be useable for multiple various anatomies with different thicknesses of the septum and/or structures of the RBB and/or LBB fibers and compatible with applicable standards (e.g., the international standard IS4 which is usable for a maximum of four independent electrical lines). This is achieved by providing an elongated pacing electrode structure with enhanced longitudinal width and reduced exposed pacing surface for low battery consumption. More specifically, in addition to a first cathode formed by the helix, a second cathode is formed by the longitudinally elongated electrode structure with reduced exposed surface for increased current density. The embodiments are directed to different options of reducing the exposed surface of the elongated electrode structure, e.g., by providing partial coverage by an insulating coating, an electrode pattern with reduced surface, and/or one or more cut-out portions in the conductive electrode surface.

The elongated second cathode allows to provide two more flexible pacing sites or sides (e.g., for concurrent LBB and RBB pacing) for various anatomies. Thereby, the multi-electrode lead is adapted to various septum wall thicknesses. To reach an effective pacing threshold, the exposed conductive surface of the elongated cathode needs to physically contact tissue and expose a limited pacing surface.

The two pacing electrodes may be electrically independent by using at least one of different timings, different thresholds, different impedances and the like.

FIG. 2 shows schematically a side view of a multi-electrode lead device 200 with a lead tip according to a first embodiment with a first cathode (C_{LV}) formed by a fixation helix 30 and a second cathode (C_{RV1}, C_{RV2}) formed by an elongated electrode structure 208a-208c with outer electrically insulating cover 208c for limited exposure.

The lead tip of the lead device 200 comprises the fixation helix 30 with an active length a that is screwed into heart tissue (septum tissue) by puncturing the tissue with the distal tip of the fixation helix 30. The lead device 200 can be used for stimulation of the LBB and RBB. The lead device 200 may for example include an elongate body that extends between a proximal end with a connector 220 that is configured to interface with an implantable pulse generator and a distal end at the fixation helix 30. The elongate body may also include a lumen that extends between the proximal end and the distal end.

In at least some of the following embodiments, as regards the design of the distal end (distality) of the lead device 200, the rate/ratio between the outer diameter of the helix 30 and the outer diameter of the housing of the lead tip may be larger than 70% ideally 100%, wherein an isoprofil distality may be provided to avoid a front stop surface for better insertion.

Furthermore, the helix 30 may be made of a rigid material to avoid deformation of the helix 30 during screwing, while a fixed helix 30 (i.e., a lock between the helix 30 and the lead body) may simplify handling (i.e., no parasite tool is required for a retractable system).

Moreover, design flexibility may be provided by adapting a distance b between the fixation helix 30 and the proximal elongated second cathode for both-side pacing and/or the longitudinal length c of the elongated second cathode to be suitable for a desired range of thicknesses of the septum in different people.

The distal design of the lead device 200 may further be configured to allow smooth and predictable advancement of the lead tip into the septum until the helix (cathode) 30 reaches the desired location at the LV chamber, i.e., close to the LBB without full perforation of the septum, so that the helix 30 does not protrude into the LV chamber.

Additionally, the design of the lead device 200 may be configured to minimize the required energy/torque to perform the puncturing of the septum. This may be achieved by providing a dedicated distally tapered lead tip (not shown) with a conical shape of an interelectrode portion between a proximal end of the helix 30 and a distal end of a proximal anode (A) 206 with an axial or longitudinal length e. The distal end of the anode 206 is located at a longitudinal distance d from the distal end of the elongated second cathode.

In certain instances, at least a proximal portion of the fixation helix 30 may be insulated and at least one turn of the distal end of the fixation helix 30 may be uninsulated. One or more turns of the fixation helix 30 (e.g., internal to the lumen of the elongate body) may be covered by a dielectric or other insulative material. The proximal portion or turn of the fixation helix 30 being eliminated may minimize impedance interference that can result from the spacing of a proximal electrode (not shown) and the fixation helix 30.

The fixation helix 30 may be mounted (e.g., welded) on a driver (not shown) which may comprise a surrounding coil or other non-flat regular or irregular surface structure (not shown) to ensure a good adhesion of the surrounding material of the lead body to the driver between proximal end of the fixation helix 30 and the distal end of the anode 206 to thereby obtain a simple rigid and durable structure of the lead tip with low number of components for improved long-term reliability. The driver (not shown) may be fixedly supported in the lead body and mechanically and electrically connected to a screwing stylet (not shown) adapter matched for insertion of a coupling end (engagement portion) of a separate screwing stylet with a screwdriver function for allowing rotational driving of the fixation helix 30 via the driver. Due to an electrical connection between the fixation helix 30 and the screwing stylet, electrical signals sensed by the fixation helix 30 at the target area can be routed via the screwing stylet to a signal analyzer and used for monitoring correct placement of the fixation helix 30 during the screwing operation without any disconnection, allowing a single step operation.

The conical shape of the lead tip may for example be based on dimensional parameters of an outer diameter Da of the proximal anode 206, an outer diameter Dl of the distal section of the lead tip, an outer diameter Dh of the fixation helix 30, the length a of the fixation helix 30, and total length Lt (e.g., e+d+b) of the lead tip including the fixation helix 30 and a tapered portion of the lead body (e.g., surrounding the driver) between the fixation helix 30 and the proximal anode 206.

In examples, the rate/ratio Dh/Dl can be set between 0.8 and 1, while Dh can be set between 1 and 1.55 mm (preferably 1.40 mm). Da can be set between 1.25 mm and 1.94 mm (preferably 1.66 mm), the length a of the fixation helix 30 can be set between 2 and 5 mm. The difference b−a can be set between 5 and 7 mm, the longitudinal length c of the elongated second cathode can be set between 7 and 11 mm, the distance d can be set between 10 and 20 mm, and the longitudinal length e of the anode 206 can be set between 5 and 10 mm.

The proposed specific conical shape with the above-mentioned ranges of dimension ensures that the lead tip with the fixation helix 30 can be used to puncture tissue in the target area in a controlled and smooth manner providing a conic profile minimizing the required energy to perform the puncture.

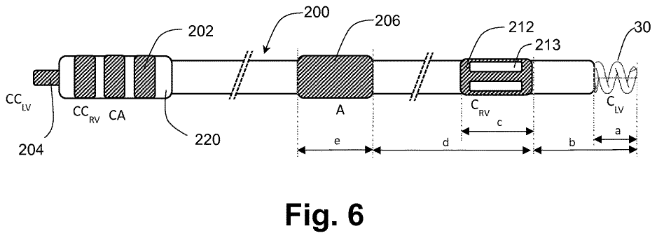

In the specific example shown in FIG. 2 and subsequent FIGS. 4 and 6, the connector 220 at the proximal end of the lead device 200 comprises four independent connector electrodes (terminals) including three circumferential connector electrodes 202 and one axial connector electrode 204. This connector design corresponds to the standard IS4 connector and is configured to provide connections for a maximum of four electrical lines or wires of the lead device 200. In the specific example, three of the four connector electrodes are used for connecting to the anode 206, the elongated second cathode and the first cathode at the fixation helix 30. The axial connector electrode 204 corresponds to a first connector electrode (CC_{LV}) 204 that connects via a first line to the fixation helix 30 (cathode C_{LV} for LV pacing). A first one (CC_{RV}) of the circumferential connector electrodes 202 connects via a second line to the elongated second cathode (C_{RV1}, C_{RV2} for RV pacing). Finally, a second one (CA) of the circumferential connector electrodes 202 connects via a third line to the anode (A).

The anode 206 can be used as a sensing electrode with a large surface (e.g., 40 mm²) and may be structures as a single electrode or two electrodes.

In the embodiment of FIG. 2, the elongated second cathode is structured as one single long and optionally flexible cathode that is connected via a single inner (internal) connection to a wire or line that connects to the most proximal one of the three circumferential connector electrodes 202 of the connector 220. Thereby, a simple and robust design can be achieved.

The elongated second cathode comprises a first ring-shaped exposed area 208a at the proximal end of the elongated second cathode and a second ring-shaped exposed area 208b at the distal end of the elongated second cathode. In an example, the total exposed area of the two ring-shaped exposed areas 208a and 208b may sum up to a value from 4 mm² to 8 mm². To reduce the total size of the exposed area of the elongated second cathode, a central portion of the elongated second cathode is coated with the electrically insulating cover 208c.

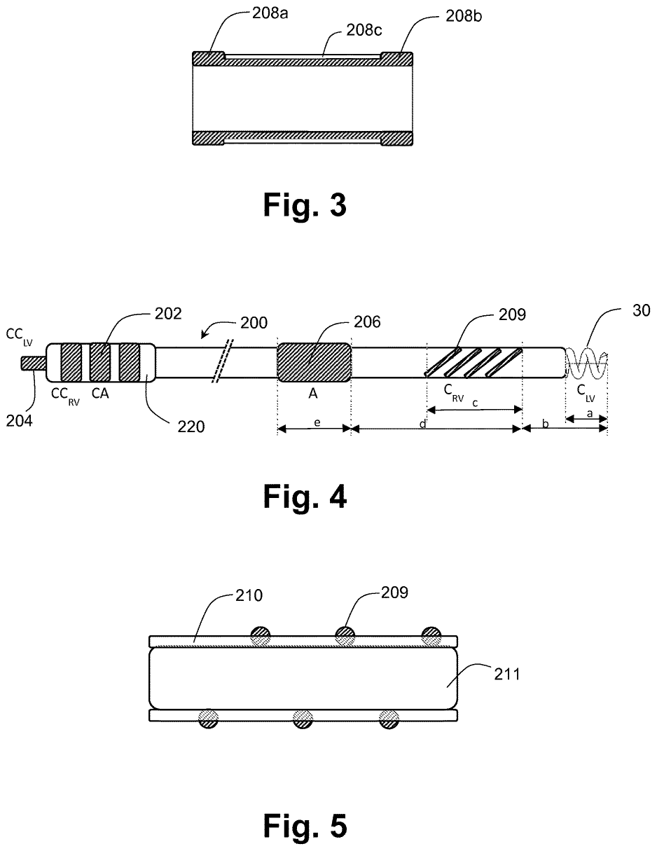

FIG. 3 shows a cross sectional view of the elongated electrode structure of the first embodiment.

The elongated second cathode is made as a single mechanical component from a conductive material of a cylindrical shape. It comprises a recessed portion in the insulated central section, in which the electrically insulating cover 208c is embedded by depositing an isolating material (e.g., a parylene coating or other insulating coating). Thereby, the two exposed cathode areas (cathodes) 208a and 208b are obtained, while both cathodes are connected to the same inner wire to simplify the internal design and provide a robust electrode structure. The reduced exposed area increases the current density during pacing and thereby allows reduced energy/battery consumption.

FIG. 4 shows schematically a side view of a multi-electrode lead device according to a second embodiment with a first cathode (C_{LV}) formed by a fixation helix 30 and a second cathode (C_{RV}) 209 formed by an elongated helical electrode structure with the longitudinal length c wound around a body of the lead device 200 for limited exposure.

The elongated second cathode 209 is connected via a single internal wire or line to the most proximal one of the three circumferential connector electrodes 202. The helical electrode structure of the elongated second cathode provides a larger longitudinal length c for better adaptation to different septum anatomies while reducing the exposed surface for reduced energy/battery consumption. It may be patterned as a single coil or multiple coils connected together.

FIG. 5 shows a cross sectional view of the elongated helical electrode structure of the second embodiment.

The lead body 211 of the lead device 200 may be coated with an isolating coating 210 (e.g., silicone or polyurethane backfilling) to decrease the exposed surface (pacing surface) and a helical pacing coil (or other pattern) 209 is placed around or partially embedded in the isolating coating 210. Thereby, a single long and flexible second cathode is obtained, that can be connected via a single inner connection to a wire or line that connects to the connector 220 to achieve a simple and robust design. In an example, the total exposed surface of the helical elongated second cathode 209 may range from 4 to 8 mm² with a total length ranging from 2 to 10 mm.

FIG. 6 shows a side view of a multi-electrode lead device according to a third embodiment with a first cathode formed by a fixation helix 30 and an elongated second cathode 212 formed by an elongated electrode structure with cut-out portions 213 for limited/reduced exposure.

Again, the elongated second cathode 212 can be made as a single mechanical component from a conductive material of a cylindrical shape. It comprises the cut-out portions 213 that may be open (without conductive material) or filled with an insulating material to reduce the total size of the exposed surface and thereby increase the current density for lower energy/battery consumption.

The cut-out portions may have an elongated rectangular shape and may be arranged at substantially equal distances around the circumference of the elongated second cathode 212. As an alternative, other shapes (oval, circular, slit-like, etc.) may be provided and/or more than one cut-out portion may be provided in the axial direction. As another alternative, smaller cut-out portions of same of different shapes may be spread over the surface of the elongated second cathode 212. As a further alternative, one or more cut-out portions may be arranged to extend in a circumferential direction of the elongated second cathode 212 to cover a major part or different angular portions of the circumference of the elongated second cathode 212.

Thereby, the total exposed area of the elongated second cathode 212 can be reduced, while the elongated second cathode 212 is connected to a single inner wire or line to simplify the internal design and provide a robust electrode structure. The reduced exposed area increases the current density during pacing and thereby allows reduced energy/battery consumption.

As an example which is applicable to all above embodiments, the elongated second cathode may be configured so that a limited size of the active electrode surface remains, which ranges between 2 and 8 mm².

The electrode pattern(s) of the elongated surface electrode can be produced by a selective insulation coverage (e.g., parylene coating which provides high insulation and good biocompatibility) achieved by e.g. a mechanical masking of the surface of the limited pacing surface that shall remain and/or by (numerically controlled) selective laser ablation of a full coated electrode surface (locally removing the insulation coverage) and/or by laser texturing to add surface material.

To summarize, multi-electrode lead devices that comprise an interelectrode portion between a distal first electrode and a proximal second electrode have been described, wherein the interelectrode portion comprises an elongated surface electrode for right bundle branch pacing, the elongated surface electrode having a reduced exposed area and at least one non-exposed portion to obtain an increased current density, wherein the elongated surface electrode is connected to a single pacing input terminal of a proximal lead connector. The elongated surface electrode with reduced exposed area enables lower energy consumption by achieving the increased current density and provides an enhanced right bundle branch pacing area for various anatomies.

While the invention has been illustrated and described in detail in the drawings and foregoing description, such illustration and description are to be considered illustrative or exemplary and not restrictive. The invention is not limited to the disclosed embodiments. It can be applied to various types of lead devices (e.g., bradycardia or tachycardia lead devices with multi-lumen, coaxial or coradial structure) and applications in the field of cardiac pacing or sensing systems.

The proposed multi-electrode lead device 200 with elongated electrode structure may be configured to be adapted or adaptable to IS1, IS4 (low voltage) or DF4 (high voltage) connectors. It can be used in connection with leadless pacemakers that would have the elongated second cathode in contact with the RV septal branch and the first cathode (helix) able to go through the septum so has to reach the LBB and deliver a synchronized pacing pulses on both ventricles. Thereby, no additional hardware is required.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. The foregoing description details certain embodiments of the invention. It will be appreciated, however, that no matter how detailed the foregoing appears in the text, the invention may be practiced in many ways, and is therefore not limited to the embodiments disclosed. It should be noted that the use of particular terminology when describing certain features or aspects of the invention should not be taken to imply that the terminology is being re-defined herein to be restricted to include any specific characteristics of the features or aspects of the invention with which that terminology is associated.

1. A lead device (200) for left and/or right bundle branch pacing, the lead device (200) comprising:
a distal first electrode (30) configured to be inserted into a septum of a heart by puncturing and to be used for left bundle branch pacing;
a proximal second electrode (206); and
an interelectrode portion between the distal first electrode (30) and the proximal second electrode (206);
wherein the interelectrode portion comprises an elongated surface electrode (208a-c; 209; 212) for right bundle branch pacing, the elongated surface electrode (208a-c;
209; 212) having a reduced exposed area (208a, 208b) and at least one non-exposed portion (208c; 213); and
wherein the elongated surface electrode (208a-c; 209; 212) is connected to only one pacing input terminal of a proximal lead connector (220).

2. The lead device (200) of claim 1, wherein the non-exposed portion comprises an electrically insulating cover (208c).

3. The lead device (200) of claim 2, wherein the electrically insulating cover (208c) is arranged in a recess of the elongated surface electrode (208a-c).

4. The lead device (200) of claim 1, wherein the elongated surface electrode comprises a helical electrode pattern (209) wound around a lead body (211) of the lead device (200).

5. The lead device (200) of claim 4, wherein the helical electrode pattern (209) is placed around or at least partially embedded in an isolating coating (210) of the lead body (211).

6. The lead device (200) of claim 1, wherein the non-exposed portion comprises at least one cut-out portion (213).

7. The lead device (200) of claim 6, wherein a plurality of the cut-out portions (213) are distributed over the surface of the elongated surface electrode (212).

8. The lead device (200) of any one of the preceding claims, wherein the distal first electrode is formed by a fixation helix (30).

9. The lead device (200) of any one of the preceding claims, wherein the proximal second electrode is an anode (206).

10. The lead device (200) of claim 8, wherein a ratio between a first outer diameter of the fixation helix (30) and a second outer diameter at a distal end of the interelectrode portion is set between 0.8 and 1, the first outer diameter is set between 1 and 1.8 mm, the length of the elongated surface electrode (208 a-c; 209; 212) is set between 7 and 11 mm, and the length of the fixation helix (30) is set between 1.5 and 5 mm.

11. The lead device (200) of any of the preceding claims, wherein the interelectrode portion has a conical shape.

12. The lead device (200) of any of the preceding claims, wherein the body (211) of the lead device (200) has a coradial structure.

13. The lead device (200) of any of the preceding claims, wherein the axial distance between the distal end of the fixation helix (30) and the distal end of the elongated surface electrode (208 a-c; 209; 212) ranges from 7 to 12 mm, the axial length of the elongated surface electrode (208 a-c; 209; 212) ranges from 7 to 11 mm, and the axial distance between the distal end of the elongated surface electrode (208a-c; 209; 212) and the distal end of the proximal second electrode (206) ranges from 10 to 20 mm.

14. The lead device (200) of any of the preceding claims, wherein the elongated surface electrode (208a-c; 209; 212) is configured to obtain a size of a resultant limited active electrode surface within a range of 2 to 8 mm².
